SEQUENCE LISTING (3 sequences)

SEQ ID NO: 1 - protein, 993 aa, Homo sapiens [sequence omitted]

SEQ ID NO: 2 - protein, 15 aa, Homo sapiens
  MOD_RES (4)..(4): PHOSPHORYLATION; tyrosine at position 4 is phosphorylated
PHTYQNRRPFSREMC

SEQ ID NO: 3 - protein, 15 aa, Homo sapiens
  MOD_RES (11)..(11): PHOSPHORYLATION; tyrosine at position 11 is phosphorylated
CGRVSEAPHTYQNRR

(19) United States
(12) Patent Application Publication
(10) Pub. No.: US 2010/0093008 A1
(43) Pub. Date: Apr. 15, 2010

(54) PHOSPHO-SPECIFIC ANTIBODIES TO FLT3 (TYR969) AND USES THEREOF

(75) Inventors: Valerie GOSS, Seabrook, NH (US); Roberto Polakiewicz, Lexington, MA (US); Kimberly Lee, Seattle, WA (US); Ting-Lei Gu, Woburn, MA (US); Albrecht Moritz, Salem, MA (US); Jiong Wu, Reading, MA (US)

Correspondence Address: Nancy Chiu Wilker, Ph.D.;Chief Intellectual Property Counsel, CELL SIGNALING TECHNOLOGY, INC., 3 Trask Lane, Danvers, MA, 01923, US

(73) Assignee: CELL SIGNALING TECHNOLOGY, INC.

(21) Appl. No.: 11/681,521

(22) Filed: Mar. 2, 2007

(51) Int. Cl. C12Q 1/48; C07K 16/40; C12N 5/12

(52) U.S. Cl. 435/15; 530/389.1; 530/388.1; 435/346

(57) ABSTRACT

The invention discloses a newly discovered Flt3 phosphorylation site, tyrosine 969 (Tyr969) in the intracellular domain, and provides reagents, including polyclonal and monoclonal antibodies, that selectively bind to Flt3 when phosphorylated at this site. Also provided are assays utilizing this reagent, including methods for determining the phosphorylation of Flt3 in a biological sample, selecting a patient suitable for Flt3 inhibitor therapy, profiling Flt3 activation in a test tissue, and identifying a compound that modulates phosphorylation of Flt3 in a test tissue, by using a detectable reagent, such as the disclosed antibody, that binds to Flt3 only when phosphorylated at Tyr969. The sample or test tissue may be taken from a subject suspected of having cancer, such as acute myelogenous leukemia (AML).

### RELATED APPLICATIONS

This application claims priority to and the benefit of PCT/US06/00979, filed Jan. 12, 2006, presently, which itself claims priority to U.S. Ser. No. 60/651,583, filed Feb. 10, 2005, now abandoned, the disclosures of which are hereby incorporated herein in their entirety.

### FIELD OF THE INVENTION

The invention relates generally to antibodies, and more particularly to activation state-specific antibodies to receptor tyrosine kinases and their uses.

### BACKGROUND OF THE INVENTION

Many cancers are characterized by disruptions in cellular signaling pathways that lead to uncontrolled growth and proliferation of cancerous cells. Receptor tyrosine kinases (RTKs) play a pivotal role in these signaling pathways, transmitting extracellular molecular signals into the cytoplasm and/or nucleus of a cell. Cells of virtually all tissue types express transmembrane receptor molecules with intrinsic tyrosine kinase activity through which various growth and differentiation factors mediate a range of biological effects (reviewed in Aaronson, Science 254: 1146-52 (1991)). RTKs share a similar architecture, having an intracellular catalytic domain, a hydrophobic transmembrane domain, and an extracellular ligand-binding domain. The binding of ligand to the extracellular portion is believed to promote dimerization, resulting in trans-phosphorylation and activation of the intracellular tyrosine kinase domain (see Schlessinger et al., Neuron 9: 383-391 (1992)).

Biological relationships between various human malignancies and disruptions in growth factor-RTK signal pathways are known to exist. For example, overexpression of EGFR-family receptors is frequently observed in a variety of aggressive human epithelial carcinomas, such as those of the breast, bladder, lung and stomach (see, e.g., Neal et al., Lancet 1: 366-68 (1985); Sainsbury et al., Lancet 1: 1398-1402 (1987)). Similarly, overexpression of HER2 has also been correlated with other human carcinomas, including carcinoma of the stomach, endometrium, salivary gland, bladder, and lung (see, e.g. Yokota et al., Lancet 1: 765-67 (1986); Fukushigi et al., Mol. Cell. Biol. 6: 955-58 (1986)). Phosphorylation of such RTKs activates their cytoplasmic domain kinase function, which in turns activates downstream signaling molecules. RTKs are often phosphorylated at multiple different sites, such as distinct tyrosine residues. These enzymes are gaining popularity as potential drug targets for the treatment of cancer. For example, Iressa™, an inhibitor of EGFR, has recently entered clinical trials for the treatment of breast cancer.

FMS-related tyrosine kinase 3 (Flt3) is a receptor tyrosine kinase preferentially expressed in hematopoietic progenitor cells. The sequence for the human Flt3 gene has been published (see Small et al., Blood 15(4): 1110-9 (1993)). It has previously been shown that Flt3 is phosphorylated at tyrosine 958 in the C terminal domain (see Casteran et al., Cell Mol. Biol. 40(3): 443-56 (1994); Beslu et al., J. Biol. Chem. 271: 20075-81 (1996)). Recent studies have indicated that the Flt3 gene is mutated by internal tandem duplication in 20-25% of adults with acute myelogenous leukemia (AML), leading to phosphorylation and overactivation of Flt3 activity in cancerous cells (see Whitman et al., Cancer Res. 61(19): 7233-39 (2001) Kottardis et al., Blood 98(6): 1752-59 (2001)). AML is the most common type of leukemia in adults, with an estimated 10,000 new cases annually (source: The Leukemia & Lymphoma Society (2001)). Flt3 has also been implicated in neural-crest derived tumors and myelodysplastic syndromes (see Timeus et al., Lab Invest. 81(7): 1025-37 (2001); Zwierzina et al., Leukemia 13(4): 553-57 (1999)). Mutation of Flt3 at aspartic acid 835 (asp835) has been implicated in progression of AML (see Abu-Duhier et al., Br. J. Haematol. 113(4): 983-88 (2001)). Although patient risk of AML may be clinically detected by examining genetic mutation of the Flt3 gene, many diagnoses are not made until patients present with symptoms of the disease, such as easy bruising, anemia and fatigue, or low white cell count. In addition, activation of the Flt3 receptor kinase leading to AML may occur in the absence of genetic mutations of the Flt3 gene.

Inhibitors of Flt3 are presently being studied as potential AML therapeutics (see Naoe et al., Cancer Chemother. Pharmacol. 48: Suppl. 1: S27-30 (2001)). For example, agonist antibodies that bind the extracellular domain of Flt3 and activate its tyrosine kinase activity have been described (see U.S. Pat. No. 5,635,388, Bennett et al.). More recent results indicate that Flt3 inhibitors have anti-tumor activity in pre-clinical models (Weisberg et al., Cancer Cell 1(5): 433-43 (2002); Kelly et al., Cancer Cell 1(5): 421-32 (2002)). However, Flt3 expression alone does not always correlate with patient response (personal communication, Dr. Donald Small, Johns Hopkins University). A limited number of Flt-3 specific antibodies, including those to tyrosines 589 and 591, are commercially available (see Cell Signaling Technology, Inc. 2005-06 Catalog p. 266-267, Cat. Nos. 3461, 3464).

Accordingly, new and improved reagents for the detection of Flt3 activity would be desirable, including development of reagents against newly identified sites of Flt3 phosphorylation. Since phosphorylation-dependent over-activation of Flt3 is associated with diseases such as AML, reagents enabling the specific detection of Flt3 activation would be useful tools for research and clinical applications.

### SUMMARY OF THE INVENTION

The invention discloses a novel Flt3 phosphorylation site, tyrosine 969 (Tyr969) in the intracellular domain, and provides antibodies, both polyclonal and monoclonal, which selectively bind to Flt3 only when phosphorylated at this novel site. Also provided are methods of determining the phosphorylation of Flt3 in a biological sample, identifying a patient suitable for Flt3 inhibitor therapy, profiling Flt3 activation in a test tissue, and identifying a compound that modulates phosphorylation of Flt3 in a test tissue, by using a detectable reagent, such as the disclosed antibodies, that binds to Flt3 when phosphorylated at Tyr969. In preferred embodiments, the sample or test tissue is taken from a subject suspected of having cancer, such as AML, characterized by or involving Flt3 activity.

### BRIEF DESCRIPTION OF THE DRAWINGS

FIG. 1—is the amino acid sequence (1-letter code) of human Flt3 (SEQ ID NO: 1). Tyrosine 969 is underlined; the intracellular domain comprises residues 564 to 993.

FIG. 2—is a Western blot analysis using phospho-Flt3(Tyr969) rabbit monoclonal antibody on extracts of Baf3 cells transfected with Flt-3 either not stimulated (lane 1) or stimulated with Flt3 ligand (lane 2) (top panel). The same blot was probed with FLT3 antibody showing equal amounts of FLT3 proteins were loaded on both lanes (bottom panel).

### DETAILED DESCRIPTION OF THE INVENTION

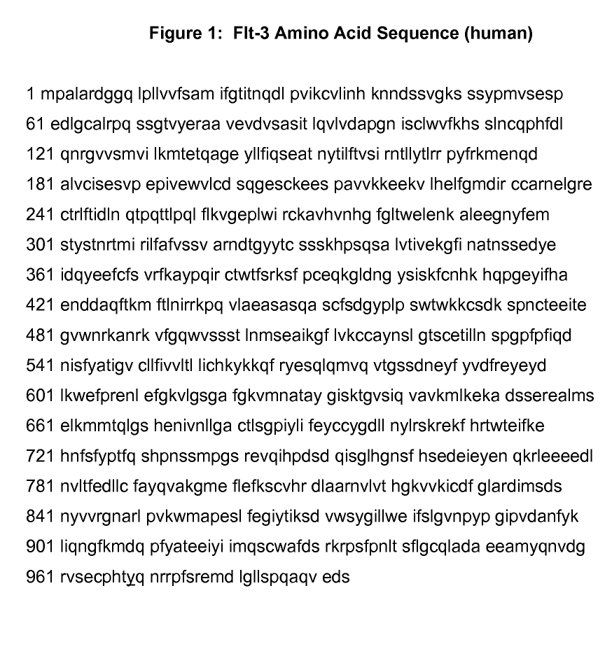

In accordance with the present invention, a novel site of Flt3 phosphorylation in the intracellular domain has now been identified, tyrosine 969 (Tyr969) (see FIG. 1). Although Flt3 phosphorylation at tyrosine 958 in the C-terminal domain has previously been described (see Beslu et al., supra.), as has phosphorylation at tyrosines 589 and 591 (see Cell Signaling Technology 2005-06 Catalog, supra.), the tyrosine phosphorylation site disclosed herein is novel. The newly identified phosphorylation site was first described by the present inventors in PCT/US06/00979 (Goss et al.) and was discovered by globally phospho-profiling human leukemia cell lines using the PhosphoScan® technique described in U.S. Patent Publication No. 20030044848, Rush et al., as further described in Example 1 herein. The phospho-profiling identified 424 novel tyrosine phosphorylation sites in a multitude of different signaling proteins, including the tyrosine 969 site in Flt-3 kinase presently described. As a result of this discovery, peptide antigens may now be designed to raise phospho-specific antibodies that bind Flt3 only when phosphorylated at Tyr969. These new reagents enable previously unavailable assays for the detection of Flt3 phosphorylation at this site.

The invention provides, in part, phospho-specific antibodies that bind to Flt3 only when phosphorylated at tyrosine 969. Also provided are methods of using a detectable reagent that binds to phosphorylated Flt3(Tyr969) to detect Flt3 phosphorylation and activation in a biological sample or test tissue suspected of containing phosphorylated Flt3 or having altered Flt3 activity, as further described below. In a preferred embodiment, the detectable reagent is a Flt3 antibody of the invention. All references cited herein are hereby incorporated herein by reference.

### A. Antibodies and Cell Lines

Flt3 phosphospecific antibodies of the present invention bind to Flt3 only when phosphorylated at Tyr969, but do not substantially bind to Flt3 when not phosphorylated at Tyr969, nor to Flt3 when phosphorylated at other tyrosine residues. The Flt3 antibodies of the invention include (a) monoclonal antibody which binds phospho-Flt3 (Tyr969), (b) polyclonal antibodies which bind to phospho-Flt3 (Tyr969), (c) antibodies (monoclonal or polyclonal) which specifically bind to the phospho-antigen (or more preferably the epitope) bound by the exemplary Flt3 (Tyr969) antibodies disclosed in the Examples herein, and (d) fragments of (a), (b), or (c) above which bind to the antigen (or more preferably the epitope) bound by the exemplary antibodies disclosed herein. Such antibodies and antibody fragments may be produced by a variety of techniques well known in the art, as discussed below. Antibodies that bind to the phosphorylated epitope (i.e., the specific binding site) bound by the exemplary Flt3(Tyr969) antibodies of the Examples herein can be identified in accordance with known techniques, such as their ability to compete with labeled Flt3 antibodies in a competitive binding assay.

The preferred epitopic site of the Flt3(Tyr969) antibodies of the invention is a peptide fragment consisting essentially of about 11 to 17 amino acids including the phosphorylated tyrosine 969, wherein about 5 to 8 amino acids are positioned on each side of the tyrosine phosphorylation site (for example, residues 963-975 of SEQ ID NO: 1).

The invention is not limited to Flt3 antibodies, but includes equivalent molecules, such as protein binding domains or nucleic acid aptamers, which bind, in a phospho-specific manner, to essentially the same phosphorylated epitope to which the Flt3 antibodies of the invention bind. See, e.g., Neuberger et al., Nature 312: 604 (1984). Such equivalent non-antibody reagents may be suitably employed in the methods of the invention further described below.

The term “antibody” or “antibodies” as used herein refers to all types of immunoglobulins, including IgG, IgM, IgA, IgD, and IgE, including F_{ab} or antigen-recognition fragments thereof. The antibodies may be monoclonal or polyclonal and may be of any species of origin, including (for example) mouse, rat, rabbit, horse, or human, or may be chimeric antibodies. See, e.g., M. Walker et al., Molec. Immunol. 26: 403-11 (1989); Morrision et al., Proc. Nat'l. Acad. Sci. 81: 6851 (1984); Neuberger et al., Nature 312: 604 (1984)). The antibodies may be recombinant monoclonal antibodies produced according to the methods disclosed in U.S. Pat. No. 4,474,893 (Reading) or U.S. Pat. No. 4,816,567 (Cabilly et al.) The antibodies may also be chemically constructed by specific antibodies made according to the method disclosed in U.S. Pat. No. 4,676,980 (Segel et al.)

The term “Flt3 antibodies” means antibodies that bind phospho-Flt3(Tyr969) both monoclonal and polyclonal, as disclosed herein. The term “does not bind” with respect to such antibodies means does not substantially react with as compared to binding to phospho-Flt3.

The term “detectable reagent” means a molecule, including an antibody, peptide fragment, binding protein domain, etc., the binding of which to a desired target is detectable or traceable. Suitable means of detection are described below.

Polyclonal antibodies of the invention may be produced according to standard techniques by immunizing a suitable animal (e.g., rabbit, goat, etc.) with an antigen encompassing pTyr(969), collecting immune serum from the animal, and separating the polyclonal antibodies from the immune serum, in accordance with known procedures. In a preferred embodiment, the antigen is a phospho-peptide antigen comprising the Flt3 sequence surrounding and including phospho-Tyr969, respectively, the antigen being selected and constructed in accordance with well known techniques. See, e.g., ANTIBODIES: A LABORATORY MANUAL, Chapter 5, p. 75-76, Harlow & Lane Eds., Cold Spring Harbor Laboratory (1988); Czernik, Methods In Enzymology, 201: 264-283 (1991); Merrifield, J. Am. Chem. Soc. 85: 21-49 (1962)). Particularly preferred peptide antigens, PHTyQNRRPFSREMC (SEQ ID NO: 2) and CGRVSEAPHTyQNRR (SEQ ID NO: 3) for Tyr969 (where y=phosphotyrosine) are described in the Examples, below. It will be appreciated by those of skill in the art that longer or shorter phosphopeptide antigens may be employed. See Id. A peptide antigen comprising phospho-Tyr969 may alternatively be employed to generate a Flt3 antibody that binds Flt3 only when phosphorylated at Tyr969. Polyclonal Flt3 antibodies produced as described herein may be screened as further described below.

Monoclonal antibodies of the invention may be produced in a hybridoma cell line according to the well-known technique of Kohler and Milstein. Nature 265: 495-97 (1975); Kohler and Milstein, Eur. J. Immunol. 6: 511 (1976); see also, CURRENT PROTOCOLS IN MOLECULAR BIOLOGY, Ausubel et al. Eds. (1989). Monoclonal antibodies so produced are highly specific, and improve the selectivity and specificity of diagnostic assay methods provided by the invention. For example, a solution containing the appropriate antigen may be injected into a mouse or other species and, after a sufficient time (in keeping with conventional techniques), the animal is sacrificed and spleen cells obtained. The spleen cells are then immortalized by fusing them with myeloma cells, typically in the presence of polyethylene glycol, to produce hybridoma cells. Rabbit fusion hybridomas, for example, may be produced as described in U.S. Pat. No. 5,675,063, C. Knight, Issued Oct. 7, 1997. The hybridoma cells are then grown in a suitable selection media, such as hypoxanthine-aminopterin-thymidine (HAT), and the supernatant screened for monoclonal antibodies having the desired specificity, as described below. The secreted antibody may be recovered from tissue culture supernatant by conventional methods such as precipitation, ion exchange or affinity chromatography, or the like.

Monoclonal Fab fragments may also be produced in Escherichia coli by recombinant techniques known to those skilled in the art. See, e.g., W. Huse, Science 246: 1275-81 (1989); Mullinax et al., Proc. Nat'l Acad. Sci. 87: 8095 (1990). If monoclonal antibodies of one isotype are preferred for a particular application, particular isotypes can be prepared directly, by selecting from the initial fusion, or prepared secondarily, from a parental hybridoma secreting a monoclonal antibody of different isotype by using the sib selection technique to isolate class-switch variants (Steplewski, et al., Proc. Natl. Acad. Sci., 82: 8653 (1985); Spira et al., J. Immunol. Methods, 74: 307 (1984)).

The invention also provides hybridoma clones, constructed as described above, that produce Flt3 monoclonal antibodies of the invention. In a preferred embodiment, the Flt3(Tyr969) monoclonal antibody of the invention is monoclonal antibody CST #C24-D9 produced by clone C24D9. Similarly, the invention includes recombinant cells producing a Flt3 antibody as disclosed herein, which cells may be constructed by well known techniques; for example the antigen combining site of the monoclonal antibody can be cloned by PCR and single-chain antibodies produced as phage-displayed recombinant antibodies or soluble antibodies in E. coli (see, e.g., ANTIBODY ENGINEERING PROTOCOLS, 1995, Humana Press, Sudhir Paul editor.)

The rabbit hybridoma cell line C24D9, which produces monoclonal antibody CST #C24-D9 that binds Flt3 only when phosphorylated at tyrosine 969 (Tyr969, was deposited with the American Type Culture Collection, in accordance with the provisions of the Budapest Treaty on ______, 2007 and has been assigned ATCC Accession Number ______.

Flt3 antibodies of the invention, whether polyclonal or monoclonal, may be screened for epitope and phospho-specificity according to standard techniques. See, e.g. Czernik et al., Methods in Enzymology, 201: 264-283 (1991). For example, the antibodies may be screened against the phospho and non-phospho peptide library by ELISA to ensure specificity for both the desired antigen (i.e. that epitope including Tyr969, respectively) and for reactivity only with the phosphorylated form of the antigen. Peptide competition assays may be carried out to confirm lack of reactivity with other Flt3 phosphoepitopes. The antibodies may also be tested by Western blotting against cell preparations containing Flt3, e.g. cell lines over-expressing Flt3, to confirm reactivity with the desired phosphorylated target. Specificity against the desired phosphorylated epitopes may also be examined by construction Flt3 mutants lacking phosphorylatable residues at positions outside the desired epitope known to be phosphorylated, or by mutating the desired phospho-epitope and confirming lack of reactivity. Flt3 antibodies of the invention may exhibit some cross-reactivity with non-Flt3 epitopes. This is not unexpected as most antibodies exhibit some degree of cross-reactivity, and anti-peptide antibodies will often cross-react with epitopes having high homology to the immunizing peptide. See, e.g., Czernik, supra. Cross-reactivity with non-Flt3 proteins is readily characterized by Western blotting alongside markers of known molecular weight. Amino acid sequences of cross-reacting proteins may be examined to identify sites highly homologous to the Flt-3 sequence surrounding Tyr969.

In certain cases, polyclonal antisera may be exhibit some undesirable general cross-reactivity to phosphotyrosine, which may be removed by further purification of antisera, e.g. over a phosphotyramine column.

Flt-3 antibodies may be further characterized via immunohistochemical (IHC) staining using normal and diseased tissues to examine Flt-3 phosphorylation and activation status in diseased tissue. IHC may be carried out according to well known techniques. See, e.g., ANTIBODIES: A LABORATORY MANUAL, Chapter 10, Harlow & Lane Eds., Cold Spring Harbor Laboratory (1988). Briefly, paraffin-embedded tissue (e.g. tumor tissue) is prepared for immunohistochemical staining by deparaffinizing tissue sections with xylene followed by ethanol; hydrating in water then PBS; unmasking antigen by heating slide in sodium citrate buffer; incubating sections in hydrogen peroxide; blocking in blocking solution; incubating slide in primary antibody and secondary antibody; and finally detecting using ABC avidin/biotin method according to manufacturer's instructions.

### B. Detection & Profiling Methods

The methods disclosed herein may be employed with any biological sample suspected of containing phosphorylated Flt3, and in particular, Flt-3 phosphorylated at Tyr969. Biological samples taken from human subjects for use in the methods disclosed herein are generally biological fluids such as serum, blood plasma, fine needle aspirate, ductal lavage, bone marrow sample or ascites fluid. In the alternative, the sample taken from the subject can be a tissue sample (e.g., a biopsy tissue), such as tumor tissue.

In one embodiment, the invention provides a method for detecting phosphorylated Flt3 in a biological sample by (a) contacting (binding) a biological sample suspected of containing phosphorylated Flt3 with at least one detectable reagent that binds to Flt3 when phosphorylated at Tyr969 in the intracellular domain under conditions suitable for formation of a reagent-Flt3 complex, and (b) detecting the presence of the complex in the sample, wherein the presence of the complex indicates the presence of phosphorylated Flt3(Tyr969) in the sample. In a preferred embodiment, the reagent is a Flt3 antibody of the invention. Biological samples may be obtained from subjects suspected of having a disease involving altered Flt3 expression or activity (e.g., leukemia or myelodysplastic syndromes), particularly cancer and AML. Samples may be analyzed to monitor subjects who have been previously diagnosed as having cancer, to screen subjects who have not been previously diagnosed as carrying cancer, or to monitor the desirability or efficacy of therapeutics targeted at Flt3. In the case of AML, for example, the subjects will most frequently be adult males and females.

In another embodiment, the invention provides a method for profiling Flt3 activation in a test tissue suspected of involving altered Flt3 activity, by (a) contacting the test tissue with at least one detectable reagent that binds to Flt3 when phosphorylated at Tyr969 in the intracellular domain under conditions suitable for formation of a reagent-Flt3 complex, (b) detecting the presence of the complex in the test tissue, wherein the presence of the complex indicates the presence of phosphorylated Flt3(Tyr969) in the test tissue, and (c) comparing the presence of phosphorylated Flt3 detected in step(b) with the presence of phosphorylated Flt3 in a control tissue, wherein a difference in Flt3 phosphorylation profiles between the test and control tissues indicates altered Flt3 activation in the test tissue. In a preferred embodiment, the reagent is a Flt3 antibody of the invention. In other preferred embodiments, the test tissue is a cancer tissue, such as an AML tissue, suspected of involving altered Flt3 Tyr969 phosphorylation.

The methods described above are applicable to examining tissues or samples from Flt3 related cancers, particularly AML, in which phosphorylation of Flt3 at Tyr969 has predictive value as to the outcome of the disease or the response of the disease to therapy. It is anticipated that the Flt3 antibodies will have diagnostic utility in a disease characterized by, or involving, altered Flt-3 activity or altered Flt-3 Tyr969 phosphorylation. The methods are applicable, for example, where samples are taken from a subject has not been previously diagnosed as having AML, nor has yet undergone treatment for AML, and the method is employed to help diagnose the disease, monitor the possible progression of the cancer, or assess risk of the subject developing such cancer involving Flt3(Tyr969) phosphorylation. Such diagnostic assay may be carried out prior to preliminary blood evaluation or surgical surveillance procedures.

Such a diagnostic assay may be employed to identify patients with activated Flt3 who would be most likely to respond to cancer therapeutics targeted at inhibiting Flt3 activity. Such a selection of patients would be useful in the clinical evaluation of efficacy of existing or future Flt3 inhibitors, as well as in the future prescription of such drugs to patients. Accordingly, in another embodiment, the invention provides a method for selecting a patient suitable for Flt3 inhibitor therapy, said method comprising the steps of (a) obtaining at least one biological sample from a patient that is a candidate for Flt3 inhibitor therapy, (b) contacting the biological sample with at least one detectable reagent that binds to Flt3 when phosphorylated at Tyr969 in the intracellular domain (SEQ ID NO: 1) under conditions suitable for formation of a reagent-Flt3 complex, and (c) detecting the presence of the complex in the biological sample, wherein the presence of said complex indicates the presence of phosphorylated Flt3(Tyr969) in said test tissue, thereby identifying the patient as suitable for Flt3 inhibitor therapy. In a preferred embodiment, the detectable reagent comprises a phospho-Flt3 antibody of the invention.

Alternatively, the methods are applicable where a subject has been previously diagnosed as having AML, and possibly has already undergone treatment for the disease, and the method is employed to monitor the progression of such cancer involving Flt3(Tyr969) phosphorylation, or the treatment thereof.

In another embodiment, the invention provides a method for identifying a compound which modulates phosphorylation of Flt3 in a test tissue, by (a) contacting the test tissue with the compound, (b) detecting the level of phosphorylated Flt3 in said the test tissue of step (a) using at least one detectable reagent that binds to Flt3 when phosphorylated at Tyr969 in the intracellular domain under conditions suitable for formation of a reagent-Flt3 complex, and (c) comparing the level of phosphorylated Flt3 detected in step(b) with the presence of phosphorylated Flt3 in a control tissue not contacted with the compound, wherein a difference in Flt3 phosphorylation levels between the test and control tissues identifies the compound as a modulator of Flt3 phosphorylation. In a preferred embodiment, the reagent is a Flt3 antibody of the invention. In other preferred embodiments, the test tissue is a taken from a subject suspected of having cancer and the compound is a Flt3 inhibitor. The compound may modulate Flt3 activity either positively or negatively, for example by increasing or decreasing phosphorylation or expression of Flt3. Flt3 phosphorylation and activity may be monitored, for example, to determine the efficacy of an anti-Flt3 therapeutic, e.g. a Flt3 inhibitor.

Conditions suitable for the formation of antibody-antigen complexes or reagent-Flt3 complexes are well known in the art (see part (d) below and references cited therein). It will be understood that more than one Flt3 antibody may be used in the practice of the above-described methods. For example, a phospho-Flt3(Tyr969) antibody of the invention and a phospho-Flt3(Tyr591) antibody may be simultaneously employed to detect phosphorylation of both tyrosines in one step.

### C. Immunoassay Formats & Diagnostic Kits

Assays carried out in accordance with methods of the present invention may be homogeneous assays or heterogeneous assays. In a homogeneous assay the immunological reaction usually involves a Flt3-specific reagent (e.g. a Flt3 antibody of the invention), a labeled analyte, and the sample of interest. The signal arising from the label is modified, directly or indirectly, upon the binding of the antibody to the labeled analyte. Both the immunological reaction and detection of the extent thereof are carried out in a homogeneous solution. Immunochemical labels that may be employed include free radicals, radioisotopes, fluorescent dyes, enzymes, bacteriophages, coenzymes, and so forth.

In a heterogeneous assay approach, the reagents are usually the specimen, a Flt3-specific reagent (e.g., the Flt3 antibody of the invention), and suitable means for producing a detectable signal. Similar specimens as described above may be used. The antibody is generally immobilized on a support, such as a bead, plate or slide, and contacted with the specimen suspected of containing the antigen in a liquid phase. The support is then separated from the liquid phase and either the support phase or the liquid phase is examined for a detectable signal employing means for producing such signal. The signal is related to the presence of the analyte in the specimen. Means for producing a detectable signal include the use of radioactive labels, fluorescent labels, enzyme labels, and so forth. For example, if the antigen to be detected contains a second binding site, an antibody which binds to that site can be conjugated to a detectable group and added to the liquid phase reaction solution before the separation step. The presence of the detectable group on the solid support indicates the presence of the antigen in the test sample. Examples of suitable immunoassays are the radioimmunoassay, immunofluorescence methods, enzyme-linked immunoassays, and the like.

Immunoassay formats and variations thereof that may be useful for carrying out the methods disclosed herein are well known in the art. See generally E. Maggio, Enzyme-Immunoassay, (1980) (CRC Press, Inc., Boca Raton, Fla.); see also, e.g., U.S. Pat. No. 4,727,022 (Skold et al., “Methods for Modulating Ligand-Receptor Interactions and their Application”); U.S. Pat. No. 4,659,678 (Forrest et al., “Immunoassay of Antigens”); U.S. Pat. No. 4,376,110 (David et al., “Immunometric Assays Using Monoclonal Antibodies”). Conditions suitable for the formation of reagent-antibody complexes are well described. See id. Monoclonal antibodies of the invention may be used in a “two-site” or “sandwich” assay, with a single cell line serving as a source for both the labeled monoclonal antibody and the bound monoclonal antibody. Such assays are described in U.S. Pat. No. 4,376,110. The concentration of detectable reagent should be sufficient such that the binding of phosphorylated Flt3 is detectable compared to background.

Flt3 antibodies disclosed herein may be conjugated to a solid support suitable for a diagnostic assay (e.g., beads, plates, slides or wells formed from materials such as latex or polystyrene) in accordance with known techniques, such as precipitation. Antibodies of the invention, or other Flt3 binding reagents, may likewise be conjugated to detectable groups such as radiolabels (e.g., ³⁵S, ¹²⁵I, ¹³¹I), enzyme labels (e.g., horseradish peroxidase, alkaline phosphatase), and fluorescent labels (e.g., fluorescein) in accordance with known techniques.

Flt3 antibodies of the invention may also be optimized for use in a flow cytometry assay to determine the activation status of Flt3 in patients before, during, and after treatment with a drug targeted at inhibiting Flt3 phosphorylation at Tyr969. For example, bone marrow cells or peripheral blood cells from patients may be analyzed by flow cytometry for Flt3 phosphorylation, as well as for markers identifying various hematopoietic cell types. In this manner, Flt3 activation status of the malignant cells may be specifically characterized. Flow cytometry may be carried out according to standard methods. See, e.g. Chow et al., Cytometry (Communications in Clinical Cytometry) 46: 72-78 (2001). Briefly and by way of example, the following protocol for cytometric analysis may be employed: fixation of the cells with 1% paraformaldehyde for 10 minutes at 37° C. followed by permeabilization in 90% methanol for 30 minutes on ice. Cells may then be stained with the primary Flt3 antibody, washed and labeled with a fluorescent-labeled secondary antibody. Alternatively, the cells may be stained with a fluorescent-labeled primary antibody. The cells would then be analyzed on a flow cytometer (e.g. a Beckman Coulter EPICS-XL) according to the specific protocols of the instrument used. Such an analysis would identify the presence of activated Flt3 in the malignant cells and reveal the drug response on the targeted Flt3 protein.

Alternatively, Flt3 antibodies of the invention may be optimized for use in other clinically-suitable applications, for example bead-based multiplex-type assays, such as IGEN, Luminex™ and/or Bioplex™ assay formats, or otherwise optimized for antibody arrays formats.

Diagnostic kits for carrying out the methods disclosed above are also provided by the invention. Such kits comprise at least one detectable reagent that binds to Flt3 when phosphorylated at Tyr969 in the intracellular domain. In a preferred embodiment, the reagent is a Flt3 antibody of the invention. In one embodiment, the diagnostic kit comprises (a) a Flt3 antibody of the invention (i.e. a phospho-specific antibody that binds phospho-Flt3(Tyr969)) conjugated to a solid support and (b) a second antibody conjugated to a detectable group. The reagents may also include ancillary agents such as buffering agents and protein stabilizing agents, e.g., polysaccharides and the like. The diagnostic kit may further include, where necessary, other members of the signal-producing system of which system the detectable group is a member (e.g., enzyme substrates), agents for reducing background interference in a test, control reagents, apparatus for conducting a test, and the like. In another embodiment a kit (e.g. a kit for the selection of a patient suitable for Flt3 inhibitor therapy) comprises (a) a Flt3 antibody as described herein, and (b) a specific binding partner (i.e. secondary antibody) conjugated to a detectable group.

The primary (phospho-Flt3(Tyr969)) detection antibody may itself be directly labeled with a detectable group, or alternatively, a secondary antibody, itself labeled with a detectable group, that binds to the primary antibody may be employed. Labels (including dyes and the like) suitable as detectable agents are well known in the art. Ancillary agents as described above may likewise be included. The test kit may be packaged in any suitable manner, typically with all elements in a single container along with a sheet of printed instructions for carrying out the test.

The following Examples are provided only to further illustrate the invention, and are not intended to limit its scope, except as provided in the claims appended hereto. The present invention encompasses modifications and variations of the methods taught herein which would be obvious to one of ordinary skill in the art.

### Example 1

### Identification of the Flt3 (Tyr969) Phosphorylation Site by Global Phospho-Profiling

In order to discover previously unknown leukemia-related signal transduction protein phosphorylation sites, PhosphoScan® peptide isolation and characterization techniques were employed to identify phosphotyrosine- and/or phosphoserine-containing peptides in cell extracts from the following human Leukemia cell lines and patient cell lines: HT-93, KBM-3, SEM, KU-812, SUP-B15, BV-173, CMK, HEL, CLL-220, CLL-1202, CLL23LB4, MEC1, MEC2, MO1043, K562, EOL1, HL60, CTV-1, REH, MV4-11, PL-21, and MKPL-1; or from the following cell lines expressing activated BCR-Abl wild-type and mutant kinases such as: Baf3-p210 BCR-Abl, Baf3-M351 T-BCR-ABL, Baf3-E255K-BCR-Abl, Baf3-Y253F-BCR-Abl, Baf3-T315I-BCR-ABl, 3T3-v-Abl; or activated Flt3 kinase such as Baf3-FLT3. This work was first described by the present inventors in PCT/US06/00979 (Goss et al.), the disclosure of which is hereby incorporated herein in its entirety by reference.

Tryptic phosphotyrosine- and phosphoserine-containing peptides were purified and analyzed from extracts of each of the 29 cell lines mentioned above, as follows. Cells were cultured in DMEM medium or RPMI 1640 medium supplemented with 10% fetal bovine serum and penicillin/streptomycin. Cells were harvested by low speed centrifugation. After complete aspiration of medium, cells were resuspended in 1 mL lysis buffer per 1.25×10⁸ cells (20 mM HEPES pH 8.0, 9 M urea, 1 mM sodium vanadate, supplemented or not with 2.5 mM sodium pyro-phosphate, 1 mM β-glycerol-phosphate) and sonicated.

Sonicated cell lysates were cleared by centrifugation at 20,000×g, and proteins were reduced with DTT at a final concentration of 4.1 mM and alkylated with iodoacetamide at 8.3 mM. For digestion with trypsin, protein extracts were diluted in 20 mM HEPES pH 8.0 to a final concentration of 2 M urea and soluble TLCK-trypsin (Worthington) was added at 10-20 μg/mL. Digestion was performed for 1-2 days at room temperature.

Trifluoroacetic acid (TFA) was added to protein digests to a final concentration of 1%, precipitate was removed by centrifugation, and digests were loaded onto Sep-Pak C₁₈ columns (Waters) equilibrated with 0.1% TFA. A column volume of 0.7-1.0 ml was used per 2×10⁸ cells. Columns were washed with 15 volumes of 0.1% TFA, followed by 4 volumes of 5% acetonitrile (MeCN) in 0.1% TFA. Peptide fraction I was obtained by eluting columns with 2 volumes each of 8, 12, and 15% MeCN in 0.1% TFA and combining the eluates. Fractions II and III were a combination of eluates after eluting columns with 18, 22, 25% MeCN in 0.1% TFA and with 30, 35, 40% MeCN in 0.1% TFA, respectively. All peptide fractions were lyophilized.

Peptides from each fraction corresponding to 2×10⁸ cells were dissolved in 1 ml of IAP buffer (20 mM Tris/HCl or 50 mM MOPS pH 7.2, 10 mM sodium phosphate, 50 mM NaCl) and insoluble matter (mainly in peptide fractions III) was removed by centrifugation. IAP was performed on each peptide fraction separately. The phosphotyrosine monoclonal antibody P-Tyr-100 (Cell Signaling Technology, Inc., catalog number 9411) or the phospho-motif PxpSP rabbit monoclonal antibody (Cell Signaling Technology, Inc., catalog number 2325) (pS=phosphoserine) were coupled at 4 mg/ml beads to protein G or protein A agarose (Roche), respectively. Immobilized antibody (15 μl, 60 μg) was added as 1:1 slurry in IAP buffer to 1 ml of each peptide fraction, and the mixture was incubated overnight at 4° C. with gentle rotation. The immobilized antibody beads were washed three times with 1 ml IAP buffer and twice with 1 ml water, all at 4° C. Peptides were eluted from beads by incubation with 75 μl of 0.1% TFA at room temperature for 10 minutes.

Alternatively, one single peptide fraction was obtained from Sep-Pak C18 columns by elution with 2 volumes each of 10%, 15%, 20%, 25%, 30%, 35% and 40% acetonitirile in 0.1% TFA and combination of all eluates. IAP on this peptide fraction was performed as follows: After lyophilization, peptide was dissolved in 1.4 ml IAP buffer (MOPS pH 7.2, 10 mM sodium phosphate, 50 mM NaCl) and insoluble matter was removed by centrifugation. Immobilized antibody (40 μl, 160 μg) was added as 1:1 slurry in IAP buffer, and the mixture was incubated overnight at 4° C. with gentle shaking. The immobilized antibody beads were washed three times with 1 ml IAP buffer and twice with 1 ml water, all at 4° C. Peptides were eluted from beads by incubation with 55 μl of 0.15% TFA at room temperature for 10 min (eluate 1), followed by a wash of the beads (eluate 2) with 45 μl of 0.15% TFA. Both eluates were combined.

### Analysis by LC-MS/MS Mass Spectrometry.

40 μl or more of IAP eluate were purified by 0.2 μl StageTips or ZipTips. Peptides were eluted from the microcolumns with 1 μl of 40% MeCN, 0.1% TFA (fractions I and II) or 1 μl of 60% MeCN, 0.1% TFA (fraction III) into 7.6 μl of 0.4% acetic acid/0.005% heptafluorobutyric acid. This sample was loaded onto a 10 cm×75 μm PicoFrit capillary column (New Objective) packed with Magic C18 AQ reversed-phase resin (Michrom Bioresources) using a Famos autosampler with an inert sample injection valve (Dionex). The column was then developed with a 45-min linear gradient of acetonitrile delivered at 200 nl/min (Ultimate, Dionex), and tandem mass spectra were collected in a data-dependent manner with an LCQ Deca XP Plus ion trap mass spectrometer essentially as described by Gygi et al., supra.

### Database Analysis & Assignments.

MS/MS spectra were evaluated using TurboSequest in the Sequest Browser package (v. 27, rev. 12) supplied as part of BioWorks 3.0 (ThermoFinnigan). Individual MS/MS spectra were extracted from the raw data file using the Sequest Browser program CreateDta, with the following settings: bottom MW, 700; top MW, 4,500; minimum number of ions, 20; minimum TIC, 4×10⁵; and precursor charge state, unspecified. Spectra were extracted from the beginning of the raw data file before sample injection to the end of the eluting gradient. The IonQuest and VuDta programs were not used to further select MS/MS spectra for Sequest analysis. MS/MS spectra were evaluated with the following TurboSequest parameters: peptide mass tolerance, 2.5; fragment ion tolerance, 0.0; maximum number of differential amino acids per modification, 4; mass type parent, average; mass type fragment, average; maximum number of internal cleavage sites, 10; neutral losses of water and ammonia from b and y ions were considered in the correlation analysis. Proteolytic enzyme was specified except for spectra collected from elastase digests.

Searches were performed against the NCBI human protein database (either as released on Apr. 29, 2003 and containing 37,490 protein sequences or as released on Feb. 23, 2004 and containing 27,175 protein sequences). Cysteine carboxamidomethylation was specified as a static modification, and phosphorylation was allowed as a variable modification on serine, threonine, and tyrosine residues or on tyrosine residues alone. It was determined that restricting phosphorylation to tyrosine residues had little effect on the number of phosphorylation sites assigned.

In proteomics research, it is desirable to validate protein identifications based solely on the observation of a single peptide in one experimental result, in order to indicate that the protein is, in fact, present in a sample. This has led to the development of statistical methods for validating peptide assignments, which are not yet universally accepted, and guidelines for the publication of protein and peptide identification results (see Carr et al., Mol. Cell. Proteomics 3: 531-533 (2004)), which were followed in this Example. However, because the immunoaffinity strategy separates phosphorylated peptides from unphosphorylated peptides, observing just one phosphopeptide from a protein is a common result, since many phosphorylated proteins have only one tyrosine-phosphorylated site. For this reason, it is appropriate to use additional criteria to validate phosphopeptide assignments. Assignments are likely to be correct if any of these additional criteria are met: (i) the same sequence is assigned to co-eluting ions with different charge states, since the MS/MS spectrum changes markedly with charge state; (ii) the site is found in more than one peptide sequence context due to sequence overlaps from incomplete proteolysis or use of proteases other than trypsin; (iii) the site is found in more than one peptide sequence context due to homologous but not identical protein isoforms; (iv) the site is found in more than one peptide sequence context due to homologous but not identical proteins among species; and (v) sites validated by MS/MS analysis of synthetic phosphopeptides corresponding to assigned sequences, since the ion trap mass spectrometer produces highly reproducible MS/MS spectra. The last criterion is routinely employed to confirm novel site assignments of particular interest.

All spectra and all sequence assignments made by Sequest were imported into a relational database. Assigned sequences were accepted or rejected following a conservative, two-step process. In the first step, a subset of high-scoring sequence assignments was selected by filtering for XCorr values of at least 1.5 for a charge state of +1, 2.2 for +2, and 3.3 for +3, allowing a maximum RSp value of 10. Assignments in this subset were rejected if any of the following criteria were satisfied: (i) the spectrum contained at least one major peak (at least 10% as intense as the most intense ion in the spectrum) that could not be mapped to the assigned sequence as an a, b, or y ion, as an ion arising from neutral-loss of water or ammonia from a b or y ion, or as a multiply protonated ion; (ii) the spectrum did not contain a series of b or y ions equivalent to at least six uninterrupted residues; or (iii) the sequence was not observed at least five times in all the studies we have conducted (except for overlapping sequences due to incomplete proteolysis or use of proteases other than trypsin). In the second step, assignments with below-threshold scores were accepted if the low-scoring spectrum showed a high degree of similarity to a high-scoring spectrum collected in another study, which simulates a true reference library-searching strategy. All spectra supporting the final list of 424 assigned sequences identified (data not shown) were reviewed by at least three people to establish their credibility.

Among the 424 novel phosphorylation sites identified was the previously unknown Flt3 kinase phosphorylation site, tyrosine 969, described herein.

### Example 2

### Production of a Flt3 (Tyr969) Phosphospecific Polyclonal Antibody

15 amino acid phospho-peptide antigens, PHTyQNRRPFSREMC (SEQ ID NO: 2) and CGRVSEAPHTyQNRR (SEQ ID NO: 3) (where y=phosphotyrosine), corresponding to residues 966-979 and 960-973, respectively, of human Flt-3 (SEQ ID NO: 1) plus cysteine on the C-terminal for coupling, were constructed according to standard synthesis techniques using a Rainin/Protein Technologies, Inc., Symphony peptide synthesizer. See ANTIBODIES: A LABORATORY MANUAL, supra.; Merrifield, supra.

These peptides were coupled to KLH, and rabbits are then injected intradermally (ID) on the back with antigen in complete Freunds adjuvant (500 μg antigen per rabbit). The rabbits were boosted with the same antigen in incomplete Freund adjuvant (250 μg antigen per rabbit) every three weeks. After the fifth boost, the bleeds were collected. The sera were purified by Protein A-affinity chromatography as previously described (see ANTIBODIES: A LABORATORY MANUAL, Cold Spring Harbor, supra.). The eluted immunoglobulins are then loaded onto PHTYQNRRPFSREMC-resin and CGRVSEAPHTYQNRR-resin Knotes column. The flow through fractions were collected and applied onto the phosphorylated equivalents (Tyr969) of these resin columns. After washing the column extensively, the phospho-FLT3 (Tyr969) antibodies were eluted and kept in antibody storage buffer.

The antibody may be further tested for phospho-specificity using a Western blot assay. BaF3/FLT3 cells may be obtained from Hutchison Cancer Research Center in Seattle, Wash. BaF3/FLT3 cells are cultured in DMEM supplemented with 10% FCS and 5 U/ml IL-3. Before stimulation, the cells are starved in serum-free DMEM medium for 4 hours. The cells are then stimulated with FLT3 ligand (50 ng/ml) for 5 minutes. The cells may then be collected, washed with PBS and directly lysed in cell lysis buffer. The protein concentration of cell lysates can be measured. The loading buffer is added into cell lysate and the mixture boiled at 100° C. for 5 minutes. 20 μl (10 μg protein) of sample is added onto 7.5% SDS-PAGE gel. A standard Western blot can be performed according to the Immunoblotting Protocol set out in the Cell Signaling Technology 2005-06 Catalogue and Technical Reference, p. 415. The phospho-FLT3 (Tyr969) polyclonal antibody is used at dilution 1:1000. The results of the blots would show that the antibody, as expected, only recognizes the ˜160 kDa FLT3 activated by its ligand. It does not recognize the non-tyrosine phosphorylated FLT3 (Tyr969) in the non-stimulated cells.

In order to confirm the specificity, different cell lysates containing various tyrosine-phosphorylated RTKs may further be prepared. The Western blot assay may be performed using these cell lysates. The phospho-FLT3 (Tyr969) polyclonal antibody can be used (1:1000 dilution) to react with the different phospho-RTKs on Western blot membrane. The results would show whether the antibody, as expected, does not significantly cross-react with other highly tyrosine-phosphorylated RTKs.

### Example 3

### Production of a Flt3(Tyr969) Phosphospecific Monoclonal Antibody

A Flt3 (Tyr969) phosphospecific rabbit monoclonal antibody, C24D9, was produced from spleen cells of the immunized rabbit described in Example 2, above, following standard procedures (Harlow and Lane, 1988). The rabbit splenocytes were fused to proprietary fusion partner cells according to a proprietary protocol (see generally Loyola School of Medicine protocol (Helga Spieker-Polet) at http://www.meddean.luc.edu/lumen/DeptWebs/microbio/KNIGHT/PROTO C/Hybridom.htm.)

Colonies originating from the fusion were screened by ELISA for reactivity to the phospho-peptide and non-phospho-peptide and by Western blot analysis. Colonies found to be positive by ELISA to the phospho-peptide while negative to the non-phospho-peptide were further characterized by Western blot analysis. Colonies found to be positive by Western blot analysis were subcloned by limited dilution. Mouse ascites were produced from the single clone obtained from subcloning.

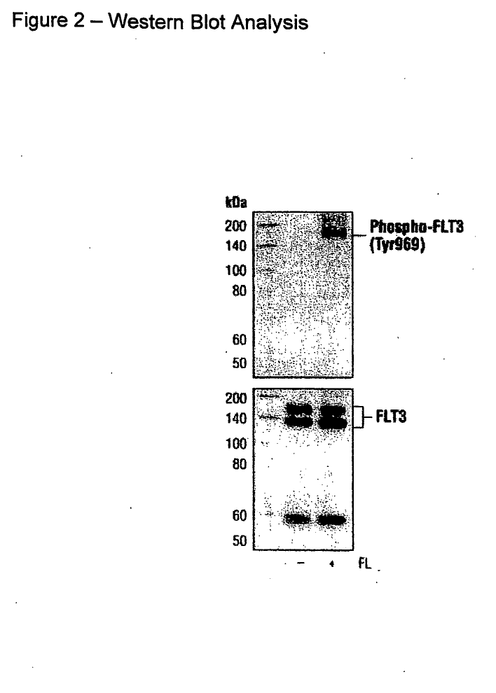

From the original fusion, 24 clones were found to be phospho-specific on ELISA. Only one of these clones was positive on Western blot analysis using cell culture supernatant, showing phospho-specificity as indicated by a strong band in the induced lane and no band in the unphosphorylated lane. This clone was subcloned to produce the C24D9 clone. Hybridoma cell culture supernatant from the single clone obtained from the Flt3 fusion was further tested by Western blot analysis. The hybridoma culture supernatant gave similar results on Western blot analysis as observed previously with the cell culture supernatant, indicating phospho-specificity on Flt3 ligand-induced Baf3 cells (FIG. 2).

### Example 4

### Detection of Flt3 Phosphorylation In Cytometric Assay

The Flt3(Tyr969) phosphospecific monoclonal antibody described in Example 3 may be used in flow cytometry to detect phospho-Flt3(Tyr969) in a biological sample, e.g. in Flt3 ligand induced Baf3 murine hematopoietic cell line. Baf3 cells that have been transfected with a Flt3 construct are serum starved for 4 hours, treated with 4 ug/ml Flt3 ligand for 5 minutes at 37° C. A sample of the cells may be taken to be analyzed by Western blot analysis. The remaining cells are fixed with 1% paraformaldehyde for 10 minutes at 37° C., followed by cell permeabilization 90% with methanol for 30 minutes on ice. The fixed cells are then stained with the Flt3(Tyr969) primary antibody for 60 minutes at room temperature. The cells are then washed and stained with an Alexa 488-labeled secondary antibody for 30 minutes at room temperature. The cells may then be analyzed on a Beckman Coulter EPICS-XL flow cytometer.

The cytometric results are expected to match the Western results described above, further demonstrating the specificity of the Flt3 (Tyr969) monoclonal antibody for the activated Flt3 protein.

### Example 5

### Detection of Constitutively Active Flt3 in Cells using Flow Cytometry

Flt3 (Tyr969) phosphospecific monoclonal antibody described above may also be used in flow cytometry to detect phospho-Flt3(Tyr969) in, e.g., the EOL-1 hematopoietic cell line that expresses an endogenous, constitutively active Flt3. Serum-starved cells may be incubated with or without the small molecule Flt3 inhibitor AG1296 for 4 hours at 37° C. The cells are then fixed with 2% paraformaldehyde for 10 minutes at 37° C. followed by cell permeabilization 90% with methanol for 30 minutes on ice. The fixed cells are stained with the Alexa 488-conjugated Flt3 primary antibody for 1 hour at room temperature. The cells may then be analyzed on a Beckman Coulter EPICS-XL flow cytometer.

The cytometric results are again expected to demonstrate the specificity of the Flt3(Tyr969) monoclonal antibody for the activated Flt3 protein and the assay's ability to detect the activity and efficacy of a Flt3 inhibitor. In the presence of the drug, a population of the cells will show less staining with the antibody, indicating that the drug is active against Flt3.

What is claimed is:

1. An antibody that binds to FMS-related tyrosine kinase 3 (Flt3) only when phosphorylated at tyrosine 969 (SEQ ID NO: 1) in the intracellular domain, but does not bind Flt3 when not phosphorylated at this position.

2. The antibody of claim 1, wherein said antibody is polyclonal.

3. The antibody of claim 1, wherein said antibody is monoclonal.

4. A hybridoma cell line producing the antibody of claim 3.

5. The hybridoma cell line of claim 4, wherein said cell line is a rabbit hybridoma or a mouse hybridoma.

6. The hybridoma cell line of claim 5, wherein said cell line is ATCC Accession No. ______.

7. A monoclonal antibody produced by the hybridoma cell line of claim 6.

8. A method for detecting phosphorylated Flt3 in a biological sample, said method comprising the steps of:
(a) contacting a biological sample suspected of containing phosphorylated Flt3 with at least one phospho-Flt3 (Tyr969) antibody of claim 1 under conditions suitable for formation of a reagent-Flt3 complex; and
(b) detecting the presence of said complex in said sample, wherein the presence of said complex indicates the presence of phosphorylated Flt3(Tyr969) in said sample.

9. The method of claim 8, wherein said biological sample is taken from a subject suspected of having cancer.

10. A method for selecting a patient suitable for Flt3 inhibitor therapy, said method comprising the steps of:
(a) obtaining at least one biological sample from a patient that is a candidate for Flt3 inhibitor therapy;
(b) contacting said biological sample with at least one phospho-Flt3
(Tyr969) antibody of claim 1 under conditions suitable for formation of a reagent-Flt3 complex; and
(c) detecting the presence of said complex in said biological sample, wherein the presence of said complex indicates the presence of phosphorylated Flt3(Tyr969) in said test tissue, thereby identifying said patient as suitable for Flt3 inhibitor therapy.

11. The method of claim 10, wherein said patient is suspected of having cancer.

12. A method for profiling Flt3 activation in a test tissue suspected of involving altered Flt3 activity, said method comprising the steps of:
(a) contacting said test tissue with at least one phospho-Flt3 (Tyr969) antibody of claim 1 under conditions suitable for formation of a reagent-Flt3 complex;
(b) detecting the presence of said complex in said test tissue, wherein the presence of said complex indicates the presence of phosphorylated Flt3(Tyr969) in said test tissue; and
(c) comparing the presence of phosphorylated Flt3 detected in step(b) with the presence of phosphorylated Flt3 in a control tissue, wherein a difference in Flt3 phosphorylation profiles between said test tissue and said control tissue indicates altered Flt3 activation in said test tissue.

13. The method of claim 12, wherein said test tissue is taken from a subject suspected of having cancer.

14. A method of identifying a compound that modulates phosphorylation of Flt3 in a test tissue, said method comprising the steps of:
(a) contacting said test tissue with said compound;
(b) detecting the level of phosphorylated Flt3 in said test tissue of step (a) using at least one phospho-Flt3 (Tyr969) antibody of claim 1 under conditions suitable for formation of a reagent-Flt3 complex;
(c) comparing the level of phosphorylated Flt3 detected in step(b) with the presence of phosphorylated Flt3 in a control tissue not contacted with said compound, wherein a difference in Flt3 phosphorylation levels between said test tissue and said control tissue identifies said compound as a modulator of Flt3 phosphorylation.

15. The method of claim 14, wherein said test tissue is taken from a subject suspected of having cancer.

16. The method of claim 15, wherein said compound is a Flt3 inhibitor.

17. A kit for the detection of phosphorylated Flt3 in a biological sample, said kit comprising (a) at least one phospho-Flt3 (Tyr969) antibody of claim 1, and (b) at least one secondary antibody conjugated to a detectable group.

18. A kit for selecting a patient suitable for Flt3 inhibitor therapy, said kit comprising (a) at least one phospho-Flt3 (Tyr969) antibody of claim 1, and (b) at least one secondary antibody conjugated to a detectable group.
